(19) United States
(12) Patent Application Publication
(10) Pub. No.: US 2023/0285836 A1
(43) Pub. Date: Sep. 14, 2023

(54) POSITION SENSE CORRECTION DEVICE, METHOD, AND PROGRAM

(71) Applicant: NIPPON TELEGRAPH AND TELEPHONE CORPORATION, Tokyo (JP)

(72) Inventor: Takashi ISEZAKI, Musashino-shi, Tokyo (JP)

(73) Assignee: NIPPON TELEGRAPH AND TELEPHONE CORPORATION, Tokyo (JP)

(21) Appl. No.: 18/011,476

(22) PCT Filed: Jun. 24, 2020
(86) PCT No.: PCT/JP2020/024817

§ 371 Date: Dec. 19, 2022

(51) Int. Cl. A63B 71/06; A63B 24/00

(52) U.S. Cl. CPC A63B 71/0622; A63B 24/0006; A63B 2024/0012

(57) ABSTRACT

A position sense correction device according to an aspect of the present invention includes a movement information obtainment unit, a storage unit, a difference calculation unit, a repetition number calculation unit, and a goal value calculation unit. The movement information obtainment unit obtains a measurement value of a physical position of a body part which a subject is able to autonomously move. The storage unit stores therein a final goal value that is set in advance. The difference calculation unit calculates a difference between the final goal value and the measurement value. The repetition number calculation unit calculates, on a basis of the difference, a number of times the subject needs to make a voluntary movement until the measurement value reaches the final goal value. The goal calculation unit calculates a goal value with respect to each voluntary movement on a basis of the number of times.

### TECHNICAL FIELD

An aspect of the present invention is related to a position sense correction device, method, and program for assisting users in having their position sense trained.

### BACKGROUND ART

Senses such as sight, smell, touch, taste, and hearing are all important. In addition, in recent years, many researches are performed on another type of sense called position sense. Position sense is the sense for determining a relative position of a part of one’s body such as an elbow, a knee, or a finger, without relying on visual angles. Similarly to the other senses, the function of one’s position sense may be degraded due to aging or a disease.

It is possible to evaluate position sense by comparing a goal joint angle that is set in advance, with an actual result of a physical movement (see Non-Patent Literature 1). To test position sense of a subject, he/she is prompted to memorize the position of a lower limb or the position of a knee joint angle used as a reference, through either visual recognition or somatosensory stimulation. After that, dependent only on memory, the subject makes a movement (a voluntary movement) to approximate a reference value of the lower limb position / the knee joint angle. From a result of the movement, it is possible to determine whether his/her position sense is normal or declining. Fortunately, even when position sense is determined to be declining, one can train his/her position sense through rehabilitation or the like (see Non-Patent Literature 2).

### CITATION LIST

### Non-Patent Literature

Non-Patent Literature 1: Nagashima, T., Okamoto, Y., and Nishimura Y., “Ichikaku kensa no undou youshiki ni yoru saigensei no chigai --- hiza kansetsu shinten kakudo ni yoru hikaku --- [Reproducibility differences caused by movement types in position sense testsComparison using knee joint extension angles --- (in Japanese) ]”, Rigaku Ryohogaku (Physical Therapy Japan)
Non-Patent Literature 2: Tahatuseikoukasyo.jp [Multiplesclerosis.jp (in Japanese)] [online] [retrieved on May 12, 2020], from the Internet <URL:https://www.tahatuseikoukasyo.jp/comfort/p001_catego ry04.html>

### SUMMARY OF THE INVENTION

### Technical Problem

Methods for training one’s position sense includes a method by which a goal body position / a goal joint angle (hereinafter, “goal value”) is presented to a subject, and the subject subsequently makes a body movement and continues performing the same task until the difference from a measured body position / a measured joint angle (hereinafter, “measurement value”) is diminished without relying of visual perception. According to this method, to begin with, the user (the subject) presented with the goal value predicts a position (hereinafter, “prediction value”) which is to be perceived as a correct answer when he/she has moved the body toward the goal value. After that, the user starts making a movement and determines that the goal value is reached when he/she feels that the difference between his/her position sense and the prediction value is minimized. During the movement, the user checks the relationship between the goal value and a current measurement value every time and, if there is a difference between the goal value and the measurement value, replans a revision scheme for the prediction value. By repeatedly performing this process, the prediction for the goal value made by the user’s position sense is revised and corrected little by little.

According to this method, however, when there is a deviation (a large difference) between the goal value and the measurement value, it is difficult to appropriately plan and carry out a revision scheme for the prediction value. In those situations, there is a possibility that a random trial-and-error process may need to be repeated. Accordingly, there is a demand for a technique that makes the training more efficient.

In view of the circumstances described above, it is an object of the present invention to provide a technique that makes it possible to train one’s position sense efficiently.

### Means for Solving the Problem

A position sense correction device according to one aspect of the present invention includes a movement information obtainment unit, a storage unit, a difference calculation unit, a repetition number calculation unit, and a goal value calculation unit. The movement information obtainment unit obtains a measurement value of a physical position of a body part which a subject is able to autonomously move. The storage unit stores therein a final goal value that is set in advance. The difference calculation unit calculates a difference between the final goal value and the measurement value. The repetition number calculation unit calculates, on a basis of the difference, a number of times the subject needs to make a voluntary movement until the measurement value reaches the final goal value. The goal calculation unit calculates a goal value with respect to each voluntary movement on a basis of the number of times.

### Effects of the Invention

According to the one aspect of the present invention, it is possible to provide a technique that makes it possible to train one’s position sense efficiently.

### BRIEF DESCRIPTION OF DRAWINGS

FIG. 1 is a block diagram showing an example of a hardware configuration of a position sense correction device 1 according to one embodiment.

FIG. 2 is a block diagram showing an example of a software configuration of the position sense correction device 1 according to the one embodiment of the present invention.

FIG. 3 is a flowchart showing an example of a processing procedure of the position sense correction device 1 according to the embodiment.

FIG. 4 is a drawing showing an example of a finger shape presented by a presentation device 1001.

### DESCRIPTION OF EMBODIMENTS

Embodiments according to the present invention will be explained below, with reference to the drawings.

### Embodiment Examples

### A Configuration

### A Hardware Configuration

At first, a configuration of a position sense correction device 1 according to one embodiment of the present invention will be explained. It is possible to use the position sense correction device 1 according to the embodiment, for example, for rehabilitation of a patient who is suffering from multiple sclerosis or the like.

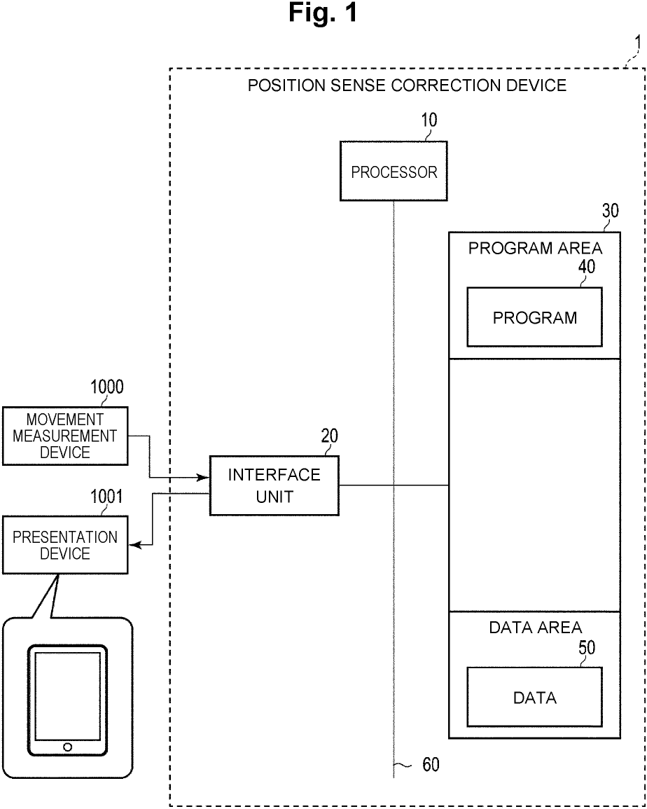

FIG. 1 is a block diagram showing an example of a hardware configuration of the position sense correction device 1 according to the one embodiment. In FIG. 1, the position sense correction device 1 is a computer including a processor 10 and a memory 30. Further, the position sense correction device 1 includes an interface unit 20. The processor 10, the memory 30, and the interface unit 20 are connected together via a bus 60.

The processor 10 may be a Central Processing Unit (CPU), a Micro Processing Unit (MPU), an Application Specific Integrated Circuit (ASIC), or an arithmetic chip such as a Field-Programmable Gate Array (FPGA).

For example, the memory 30 may include a readable/writable non-volatile memory such as a Hard Disk Drive (HDD) or a Solid State Drive (SSD), or a non-volatile memory such as a Random Access Memory (RAM), a Read Only Memory (ROM), or the like. A storage area of the memory 30 includes an area (a program area) storing therein a program 40 and another area (a data area) storing therein data 50. For example, the rest of the area is used as a stack area assigned to processes by an Operating System (OS), as a cache area, or the like.

The interface unit 20 is connectable to a movement measurement device 1000 and a presentation device 1001. Further, an input device such as a keyboard, a touch panel, a touchpad, a mouse, or the like or an output device such as a display device may also be connected to the interface unit 20. The input device retrieves operation data generated by operations of an operator. The output device outputs information to be presented to the operator, for example. Further, the interface unit 20 may be connected to a setting device so as to be provided with various types of setting data for the position sense correction device 1. Further, the interface unit 20 may also include a wired or wireless communication interface.

In FIG. 1, the movement measurement device 1000 measures movement information of a user. The measured movement information is sent to the position sense correction device 1 via a signal cable, for example,

The presentation device 1001 is a device that presents, to the user, presentation-purpose data generated by the position sense correction device 1 and is typically a tablet. The tablet includes a display device using liquid crystal or organic Electro Luminescence (EL) and an audio speaker or the like and is thus preferable as the presentation device. However, possible embodiments are not limited to this example. Possible methods for presenting information include a visual presentation using a display device, an auditory presentation using a speaker, and an electric presentation using an electrode pasted onto skin surface.

In FIG. 1, the position sense correction device 1 obtains the movement information of a user from the movement measurement device 1000. The movement information is, for example, information indicating the angle of an elbow or a knee joint, whether an arm is up or down, or whether a raised arm is the right arm or the left arm. It is possible to obtain this type of information, for example, by sensing an electromyographic signal or a muscle activity pattern through an electrode pad attached to the user. Alternatively, it is also possible to obtain the movement information through image processing performed on image data acquired by imaging a physical state of the user. In the embodiment, a measurement value of the physical position of a body part which the user is able to autonomously move will be referred to as the movement information.

In the present embodiment, with respect to the two joints (the first joint and the second joint) of a finger, an angle of each of the joints will be used as the movement information. Needless to say, the body part to be used does not have to be a finger and can be another body part. Further, physical quantities to be used are the angles (expressed one-dimensionally) of the two finger joints. However, possible embodiments are not limited to this example, and may use any angle expressed multi-dimensionally or any position expressed multi-dimensionally.

### A Software Configuration

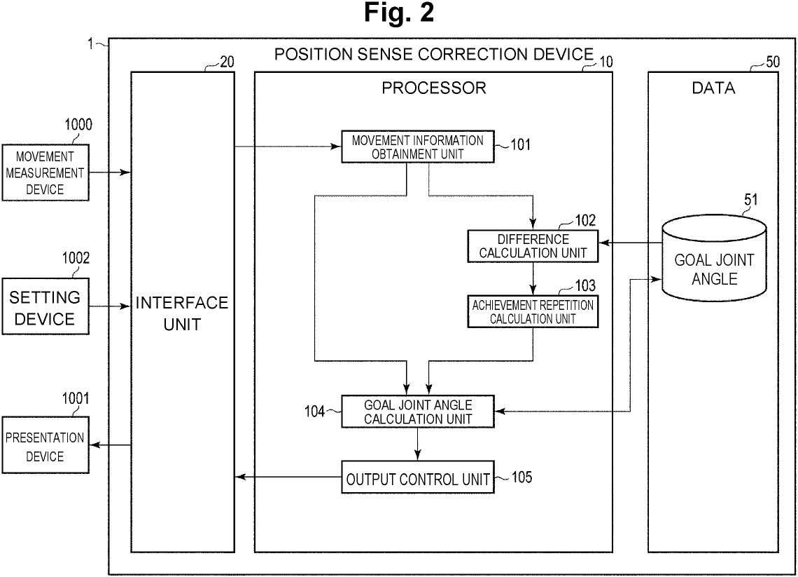

FIG. 2 is a block diagram showing a software configuration of the position sense correction device 1 according to the one embodiment of the present invention, in association with the configuration in FIG. 1.

As processing function according to the one embodiment, the processor 10 includes a movement information obtainment unit 101, a difference calculation unit 102, an achievement repetition calculation unit 103, a goal joint angle calculation unit 104, and an output control unit 105. These functional blocks are realized as a result of the processor 10 executing instructions included in the program 40. In other words, the program 40 includes the instructions for causing a computer to function as the position sense correction device 1. Further, the program 40 may be distributed as being recorded on a recording medium such as an optical recording medium or may be delivered as being downloaded via a network.

The movement information obtainment unit 101 obtains, from the movement measurement device 1000, measurement values of each of the angle of the first joint and the angle of the second joint of the index finger of the right hand. The obtained measurement values are stored into the data area of the memory 30, as the movement information. Further, the data area has stored therein a goal joint angle 51 serving as a final goal value, which is set in advance.

For example, the goal joint angle 51 is input through a setting device 1002 and is transferred to the data area via the interface unit 20. In other words, the interface unit 20 receives a setting of the goal joint angle 51 and stores the received setting into the memory. In the following sections, an example will be explained in which the goal joint angles of the first joint and the second joint are each 90°.

The difference calculation unit 102 calculates the difference between the goal joint angle 51 and the measurement values indicating the angles of the joints.

On the basis of the difference, the achievement repetition calculation unit 103 calculates the number of times the user needs to make the voluntary movement until the measurement value of the angle of each of the joints reaches the goal joint angle 51. The calculated number of times will be referred to as an “achievement repetition number”.

On the basis of the achievement repetition number, the goal joint angle calculation unit 104 calculates a goal value with respect to each of the voluntary movement of the user.

The output control unit 105 outputs the goal value of the voluntary movement to the presentation device 1001 so as to be displayed.

The following will explain the information given and received among the functional blocks. In the explanation below, the goal joint angle vector expressed as “a T and a dot” in the expressions will be written as “T(•)” in the text. In other words, Expression (1) is established. [Math. 1]

$$\text{The goal joint angle vector} \dot{T} = \text{Τ} \left(⋅\right) \qquad \text{(1)}$$

The goal joint angle 51 includes a final goal joint angle vector T input through the setting device 1002 and the goal joint angle vector T(•) sent from the goal joint angle calculation unit 104. The goal joint angle vector T(•) is obtained from the difference calculation unit 102, the achievement repetition calculation unit 103, the goal joint angle calculation unit 104, and the presentation device 1001.

Held as the final goal joint angle vector T is a vector T = [90,90] in which the goal joint angle 90° of the first joint is input as the first element, whereas the goal joint angle 90° of the second joint is input as the second element.

Similarly to the final goal joint angle vector T, the goal joint angle vector T(•) is also held as a vector in which the goal joint angle of the first joint is input as the first element, whereas the goal joint angle of the second joint is input as the second element. In an initial state, the goal joint angle vector T(•) = T is satisfied. The goal joint angle vector T(•) held in the memory 30 is updated with the most recent value calculated by the goal joint angle calculation unit 104.

The movement information obtainment unit 101 obtains movement information M from the movement measurement device 1000. The movement information M is expressed as a vector M of which the elements are the angle of the first joint and the angle of the second joint. When the angles of the two joints are both 0°, Expression (2) is true.

$$\begin{matrix}\text{M} = \\ \left[\text{The angle of the first joint, The angle of the second joint}\right] = \left[0 , 0\right]\end{matrix} \qquad \text{(2)}$$

The movement information M is sent to the difference calculation unit 102 and the goal joint angle calculation unit 104.

The difference calculation unit 102 obtains the movement information M from the movement information obtainment unit 101 and obtains the goal joint angle vector T(•) from the memory 30. Further, the difference calculation unit 102 calculates a difference “e”. It is possible to calculate the difference “e” as a norm of a difference vector between the goal joint angle vector T(•) and the movement information M, by using Expression (3), for example. [Math. 2]

$$e = \left‖\dot{T} - M\right‖ \qquad \text{(3)}$$

For example, when T (•) = [90,90] and M = [0,0] are both true, the difference “e” is calculated as presented below by using Expression (4). [Math. 3]

$$e = \left‖\left[90 , 90\right] - \left[0 , 0\right]\right‖ = \sqrt{{90}^{2} + {90}^{2}} = 90 \sqrt{2} \qquad \text{(4)}$$

The calculated difference “e” is sent to the achievement repetition calculation unit 103.

The achievement repetition calculation unit 103 obtains the difference “e” from the difference calculation unit 102 and calculates an achievement repetition number N. The achievement repetition number N can be defined as presented in Expression (5), for example, as a function f(e) of the difference “e”. [Math. 4]

$$N = f \left(e\right) \qquad \text{(5)}$$

For example, the right hand side of Expression (5) can be defined by using the pseudocode presented below including the if sentences: [Math. 5]

$$\begin{matrix}f \left(e\right) = {n}_{1} & : & i f \left(e < {s}_{1}\right) \\ = {n}_{2} & : & i f \left({s}_{1} \leq e < {s}_{2}\right) \\ ⋮ & & \\ = {n}_{k} & : & i f \left({s}_{k - 1} \leq e\right)\end{matrix}$$

where k denotes a decomposition number of the achievement repetition number N;
   Sᵢ (i = 1, ..., k-1) denotes a difference threshold value in each decomposition; and
   nᵢ (i = 1, ..., k) denotes the achievement repetition number in each decomposition.

In an example, when the decomposition number k of the achievement repetition number N satisfies k = 3, while Sᵢ = 50, S₂ = 130, nᵢ = 1, n₂ = 2, and n₃ = 3 are satisfied, Expression (6) is true. [Math. 6]

$$\begin{matrix}f \left(e\right) = 1 & : & i f \left(e < 50\right) \\ = 2 & : & i f \left(50 \leq e < 130\right) \\ = 3 & : & i f \left(130 \leq e\right)\end{matrix} \qquad \text{(6)}$$

In Expression (6), when the difference “e” satisfies e = 90√2, because f(e) = 2 is true, the achievement repetition number N is calculated as N = 2. The calculated achievement repetition number N is sent to the goal joint angle calculation unit 104.

The goal joint angle calculation unit 104 obtains the achievement repetition number N from the achievement repetition calculation unit 103, obtains the final goal joint angle vector T from the memory 30, and obtains the movement information M from the movement information obtainment unit 101. Further, the goal joint angle calculation unit 104 calculates the goal joint angle vector T(•) by using Expression (7). [Math. 7]

$$\begin{matrix}\text{Δ} M = \frac{T - M}{N} \\ \dot{T} = M + \text{Δ} M\end{matrix} \qquad \text{(7)}$$

The calculated goal joint angle vector T(•) is sent to the memory 30, so as to make an update with the most recent value.

The calculation described above is individually performed with respect to the joints in the body part being processed. On such occasion, the magnitude of the goal joint angle may be adjusted on the basis of movable ranges or interference conditions between the joints or position sense characteristics of each joint. Further, previous test data for each user may be stored so as to set the values of k, Sᵢ, and nᵢ in accordance with the results thereof. In other words, the memory 30 may store therein, with respect to each subject, a history of at least one selected from among the final goal joint angle vector T, the achievement repetition number N, and the goal joint angle vector T(•), so that the values of the parameters can be reset with reference to these pieces of data every time a test is performed.

### <An Operation>

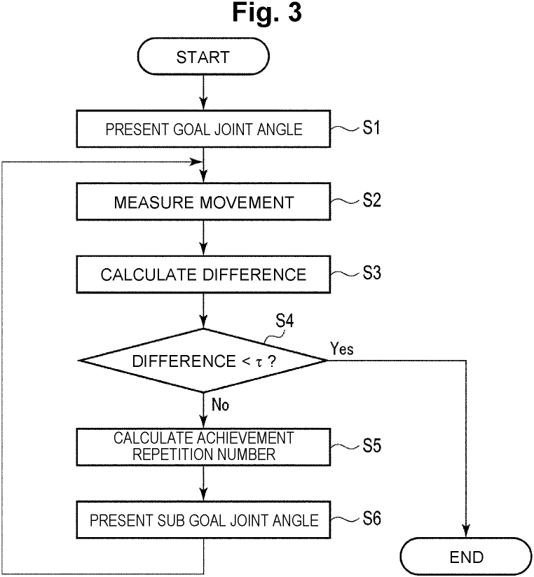

FIG. 3 is a flowchart showing an example of a processing procedure of the position sense correction device 1 according to the embodiment. A method for training position sense will be explained with reference to the flowchart. The following is based on an example in which position sense related to the first joint and the second joint of an index finger is trained.

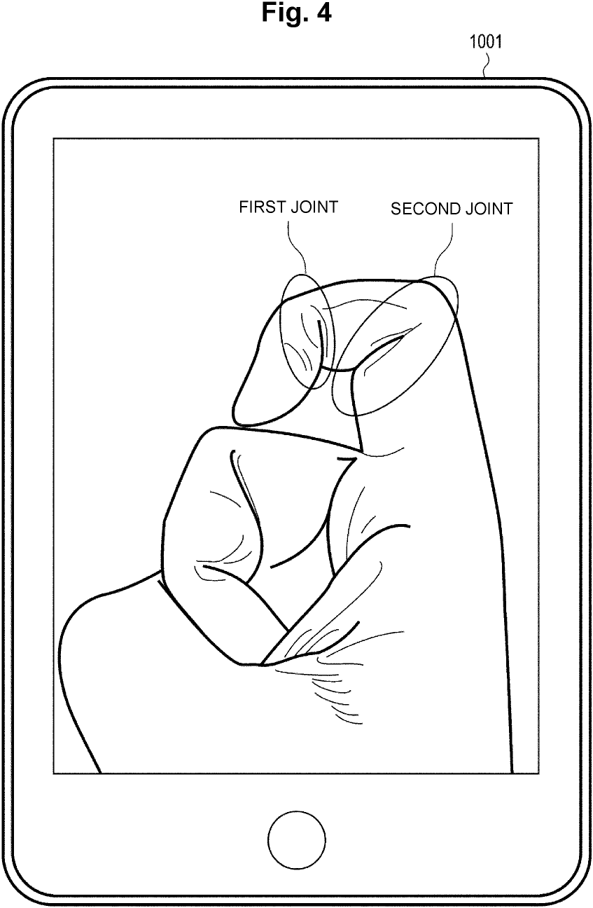

In FIG. 3, the position sense correction device 1 presents a goal joint angle to a user (step S1) . More specifically, as shown in FIG. 4, for example, finger shapes to be observed when the goal joint angle is achieved are presented. In this situation, the final goal joint angle vector T and the goal joint angle vector T(•) are stored into the memory 30.

Subsequently, a movement measurement process is performed (step S2), and the position sense correction device 1 obtains measurement values of the angles of the first and the second joints of the index finger as movement information M. After that, the position sense correction device 1 calculates a difference “e” by using Expression (3) (step S3). When the calculated difference “e” is smaller than a predetermined threshold value _{τ} (step S4: Yes), the process ends. In this situation, τ can arbitrarily be set depending on tasks or body parts.

When e < τ is not satisfied (step S4: No), the position sense correction device 1 calculates an achievement repetition number N by using Expression (5) (step S5). After that, By using Expression (7), the position sense correction device 1 calculates a goal joint angle vector T(•) and presents the calculated vector to the presentation device 1001 as the next goal value (a sub goal joint angle) (step S6).

In the processing procedure described above, the goal joint angle calculation unit 104 varies the goal joint angle vector T(•), in accordance with the perceived positions of the joints perceived by the user, i.e., the precision level of his/her position sense. Further, as the test results improve, i.e., as the precision level of his/her position sense becomes higher, the goal joint angle calculation unit 104 decreases the value of ΔM in Expression (7). In other words, the goal joint angle calculation unit 104 decreases the change amount ΔM of the goal joint angle vector T(•) .

As a result, it is possible to present the goal angle in such a manner that the difference between the goal joint angle and the actual joint angle measurement values gradually decreases in accordance with the precision levels of the user’s position sense. It is therefore possible to improve efficiency of the training.

### <Advantageous Effects>

As explained in detail above, in the one embodiment, the goal value of position sense to be aimed at by the motion of the hand or the finger of the human body is stored in the memory 30 in advance. Further, the goal value is compared with the value obtained by measuring the motion of the body part of the user, so that the sub goal value (the goal joint angle vector) to be aimed at in the next movement is calculated and presented in such a manner that the final goal value is achieved over a certain number of times that will not impose a burden. With these arrangements, the embodiment is able to provide a position sense correction device, method, and program that make it possible to train one’s position sense efficiently.

### <Other Embodiments>

(1) In the one embodiment, the configuration (FIG. 1) was explained in which the movement measurement device 1000 and the presentation device 1001 are provided separately from the position sense correction device 1. Alternatively, for example, a part of the functions of the movement measurement device 1000 and the presentation device 1001 may be installed in the position sense correction device 1.

(2) In the one embodiment, the goal joint angle 51 is input to the position sense correction device 1 through the setting device 1002 so as to be stored in the memory 30. Alternatively, for example, the goal joint angle may be obtained from an external storage medium such as a Universal Serial Bus (USB) memory or a storage device such as a database server provided in a cloud.

(3) In other examples, it is possible to apply various modifications without departing from the gist of the present invention to the configuration of the processor 10, the processing procedure, details of the processing, the user’s body part of interest (an elbow, a knee, an ankle, or other joints may be used besides the finger), the physical quantities to be measured (angles, positions, the height of a hand, the height of a foot, etc.), and the method for obtaining the measurement values.

In other words, the present invention is not strictly limited to the embodiments described above. At the stage of embodiment, it is possible to carry out the present disclosure while modifying one or more of the constituent elements without departing from the gist thereof. Further, it is also possible to form various inventions by combining together, as appropriate, two or more of the constituent elements disclosed in the above embodiments. For example, some among all the constituent elements described in the embodiments may be deleted. Further, constituent elements from mutually-different embodiments may be used in combination, as appropriate.

Reference Signs List

| | |
|---|---|
| 1 | Position sense correction device |
| 10 | Processor |
| 20 | Interface unit |
| 30 | Memory |
| 40 | Program |
| 50 | Data |
| 51 | Goal joint angle |
| 60 | Bus |
| 101 | Movement information obtainment unit |
| 102 | Difference calculation unit |
| 103 | Achievement repetition calculation unit |
| 104 | Goal joint angle calculation unit |
| 105 | Output control unit |
| 1000 | Movement measurement device |
| 1001 | Presentation device |
| 1002 | Setting device. |

1. A position sense correction device comprising:
a movement information obtainment unit that obtains a measurement value of a physical position of a body part which a subject is able to autonomously move;
a storage unit that stores therein a final goal value that is set in advance;
a difference calculation unit that calculates a difference between the final goal value and the measurement value;
a repetition number calculation unit that calculates, on a basis of the difference, a number of times the subject needs to make a voluntary movement until the measurement value reaches the final goal value; and
a goal value calculation unit that calculates a goal value with respect to each voluntary movement on a basis of the number of times.

2. The position sense correction device according to claim 1, further comprising:
an output unit that outputs the goal value to a presentation device.

3. The position sense correction device according to claim 1, further comprising:
an interface unit that receives a setting of the final goal value and stores the received setting into the storage unit.

4. The position sense correction device according to claim 1, wherein
the goal value calculation unit varies the goal value in accordance with a precision level of a perceived position of the body part perceived by the subject.

5. The position sense correction device according to claim 4, wherein
the goal value calculation unit decreases a change amount of the goal value as the precision level becomes higher.

6. The position sense correction device according to claim 1, wherein
the storage unit stores therein, with respect to each subject, a history of at least one selected from among: the final goal value, the number of times of the voluntary movement, and the goal value with respect to each voluntary movement.

7. A position sense correction method comprising:
a step of obtaining, by a computer having a memory, a measurement value of a physical position of a body part which a subject is able to autonomously move;
a step of storing, by the computer, a final goal value that is set in advance, into the memory;
a step of calculating, by the computer, a difference between the final goal value and the measurement value;
a step of calculating, by the computer, on a basis of the difference, a number of times the subject needs to make a voluntary movement until the measurement value reaches the final goal value; and
a step of calculating, by the computer, a goal value with respect to each voluntary movement on a basis of the number of times.

8. A non-transitory computer-readable medium having computer-executable instructions that, upon execution of the instructions by a processor of a computer, cause the computer to function as the position sense correction device according to claim 1.
